(19) United States
(12) Patent Application Publication
(10) Pub. No.: US 2017/0095204 A1
(43) Pub. Date: Apr. 6, 2017

(54) ORAL APPARATUS WITH RIGID SENSOR CAPTURE COMPONENT FOR STRONG COUPLING TO UPPER JAW

(71) Applicant: Wake Forest University Health Sciences, Winston-Salem, NC (US)

(72) Inventors: Joel Douglas Stitzel, JR., Winston-Salem, NC (US); Michael Avery Hurst, Winston-Salem, NC (US)

(21) Appl. No.: 15/205,791

(22) Filed: Jul. 8, 2016

Related U.S. Application Data
(60) Provisional application No. 62/189,820, filed on Jul. 8, 2015.

(51) Int. Cl. A61B 5/00; B29C 45/14; B29C 33/38; A61B 5/11; A63B 71/08

(52) U.S. Cl. CPC A61B 5/682; A61B 5/11; A63B 71/085; B29C 33/3842; B29C 45/14; A61B 2503/10; A61B 2562/0219; A61B 2560/0443; A61B 2562/12; B29L 2031/753

(57) ABSTRACT

An oral apparatus comprising a sensor or a plurality of sensors is provided for accurately measuring mechanical forces associated with concussive and sub-concussive impacts to the head, especially as in sports.

### RELATED APPLICATIONS

The present application claims priority to U.S. Provisional Patent Application No. 62/189,820, filed Jul. 8, 2015. The disclosure of U.S. Provisional Patent Application No. 62/189,820 is hereby incorporated by reference in its entirety herein.

### FIELD

The apparatus and methods relate to measuring mechanical forces on the human body, particularly for sports.

### BACKGROUND

Mechanical forces involved with traumatic brain injury (TBI) and mild traumatic brain injury (mTBI) concussions in sports have been quantified using accelerometers embedded within helmets, mostly in football and hockey (e.g., Head Impact Telemetry or HITs system). However, there is a paucity of data regarding head impacts in non-helmeted sports, particularly at the youth level. This is in part due to unreliable results from wearable sensor technology. A sensor which sticks on the skin is uncomfortable, prone to inaccuracies and lack of precision, and potentially injurious to the athlete during an impact. Headband-based sensors tend to move/shift and do not provide reproducible results. Therefore, there is a need for development of improved configurations for “wearable” non-invasive sensors that can accurately and reliably quantify mechanical forces to the head to improve understanding of the primary injury profile of concussions and to aid in the early detection of concussive injuries in all contact sports (helmeted and non-helmeted).

### SUMMARY

The invention described herein comprises an oral apparatus for quantifying accelerations and velocities as experienced by a wearer's head. The oral apparatus can include a rigid or substantially rigid central component and a sensor, and it can couple the sensor to bony portions of the upper jaw and/or teeth. The measured accelerations and velocities may be specifically associated with the upper jaw (maxilla), as representative of the larger skull.

In some embodiments, the oral apparatus for coupling a sensor to bony portions of the upper jaw (maxilla) comprises a rigid central capture component and a sensor. The apparatus may further include a resilient outer component. In some embodiments, the rigid central component is a sensor capture component. In some embodiments, the rigid central capture component firmly couples the sensor to the maxilla and/or upper teeth. Some embodiments of the oral apparatus include aspects that allow the apparatus to apply forces with respect to the maxillary anatomy to better couple the sensor with the bony structures of the upper jaw. In some embodiments the rigid central component is custom-molded for the wearer's mouth. Such a custom mold may facilitate firmer coupling and stronger adherence, for example due to mechanical locking and surface tension associated with a layer of saliva between the sensor capture component and the user's upper jaw, maxilla, gums, or teeth.

Some embodiments of the oral apparatus include a plurality of sensor or other electronic components. Some embodiments include a power source. The component arrangement or configuration can allow for contact of the tongue with the central lingual surfaces of the upper jaw. A variety of attachments can be used to secure the oral apparatus against the upper jaw. Some embodiments can include a non-corrosive sealant to protect electronic components. Some embodiments include metal, acrylic, or other rigid materials.

Some oral apparatus embodiments further include a resilient outer component. The resilient component may be an overlay, and can be permanently adhered to the rigid central component or detachable. In some embodiments, the resilient overlay is a mouthguard.

The invention may also be embodied in a method for manufacturing a custom oral apparatus for coupling a sensor to bony portions of the upper jaw, comprising: making an inverse mold for player, setting a rigid or substantially rigid central capture component configuration in the mold, incorporating a sensor capture setting into the rigid or substantially rigid capture component, and connecting (e.g., by embedding) the sensor to the rigid central component or capture setting.

### BRIEF DESCRIPTION OF FIGURES

FIG. 1 shows illustrations of the upper jaw and an oral apparatus embodiment with sensor placed laterally, in FIGS. 1A, 1B, and 1C.

FIG. 2 shows photographs of a prototype oral apparatus according to one embodiment of the invention, with sensor nearby (FIG. 2A) and captured in the capture space of the rigid central component (FIG. 2B).

FIG. 3 shows three components of an oral apparatus, according to one embodiment of the invention. FIG. 3A shows rigid central component with sensor, FIG. 3B shows resilient outer component with rigid central component inside, and FIG. 3C shows parts of the rigid central component and the resilient outer component separated.

FIG. 4 illustrates a capture component and sensor with respect to the three axes (x, y, and z) about which head movements are detected. The three axes may also be utilized to design mechanisms to secure an embodiment of the oral apparatus in place.

FIG. 5 is a photograph of an embodiment of an oral apparatus, showing a rigid central capture component comprising electronics (a sensor, power source, and wire, in this example).

FIG. 6 is a photograph of the oral apparatus embodiment of FIG. 5 placed in a maxillary mold of the user's mouth, such that electronic components are visible.

FIG. 7 is a photograph of an embodiment of an oral apparatus, including a rigid central capture component coupled with a resilient outer component.

FIG. 8 is a photograph of an embodiment of an oral apparatus, including a rigid central component coupled with a resilient outer component (at least partially visible on the external surface of the teeth), placed in a maxillary mold of the user's mouth and viewed externally, wherein only the outer component is visible, and a portion of that outer component has been cut away.

### DETAILED DESCRIPTION

Aspects and features of the present disclosure are directed to an apparatus and method for measuring forces and impacts on the skull. Preliminary work from the iTAKL (Imaging Telemetry And Kinematic modeLing in youth football) study indicates that the “cumulative” impact exposure is much more predictive/informative about the child's clinical outcome and clinical state than the “individual” impacts alone. However, these results were obtained for football using a sensor instrumentation system which is useful only for football, embedded in a helmet. One of the main barriers to exploration and understanding of other sports is instrumentation systems. Preliminary and unpublished data from several groups as well as some published data indicates that skin-based or headband/skull cap based systems are subject to lag and acceleration measurements that are not representative of the skull motion. Mouthguard based systems, largely exploratory and prototype to this point, show some promise. However, preliminary and unpublished data as well as some published data from several groups has shown that soft (resilient) mouthguard materials with embedded sensors are prone to measurement errors associated with relative motion between the sensor and the adjacent anatomy. They are also prone to chewing by athletes, low durability, can interfere with speech, and cannot be worn in situations where an athlete does not want to wear a mouthguard. Moreover, they are often incompatible with developing mouthguard technology which needs to fit over a person's existing dentition. Therefore exploiting a wearable sensor that can accurately keep track of an athlete's head impact history but can reside in the mouth is useful for the identification and early detection of concussive injuries as well as subconcussive impact measurement and impact exposure measurement.

Quantification of head impacts in non-helmeted sports can serve as an early detection tool for concussive injuries and also allow for accurate quantification of subconcussive impact exposure. A major issue with measuring these data in non-helmeted sports is coupling with the skull, which is a separate innovation from advances in wireless sensors, etc. Optimized coupling allows more accurate measurements of the forces acting on the head (skull) and the resulting accelerations, which are important for understanding what is happening to the brain.

Surprisingly, placement of a sensor (having the technical requirements needed to accurately measure these data) in a rigid capture component which is coupled with bony portions of the upper jaw, as compared to other sensor configurations and/or positions associated with the skull, generated preliminary results suggesting more accurate velocity and acceleration measurements. Mouthguards designed with resilient but softer coupling components are used because soft materials are better for covering and protecting teeth. However, these can be associated with durability issues due to chewing by athletes, interference with speech and breathing, and also bulk when sensors are placed outside the teeth. The ability to speak well with a retainer style design instrumentation in the mouth is also an unexpected result, which is confirmed with preliminary data in children and adults. Although mouthguards provide close proximity to the dentition and the skull, embedding of sensor and electronic components in the soft material of a mouthguard is associated with an unexpected amount of sensor motion during impacts which creates some difficulties with accuracy and precision of acceleration measurements. The specific technique employed herein provided unexpectedly accurate measurements of velocity and acceleration associated with less oscillation and noise in the signal, providing better precision and accuracy.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application and business-related constraints, and that these specific goals will vary from one implementation to another and from one developer to another.

### DEFINITIONS

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and all three of A and B and C.

As used herein, a “substantially rigid central component,” a “rigid central component” or a “central capture component” refers to a central unit of a hard or firm material in an oral apparatus. The component is made from a rigid material that couples well with the palate of the upper jaw, especially when a layer of saliva is present. The rigid central component comprises a sensor and optionally other components, such as electrical components.

As used herein, a “resilient outer component” or “resilient component” refers to an optional layer of a comparatively softer or more pliable material than the rigid component material. The resilient outer component can be an additional layer over the rigid central component.

As used herein, a “sensor capture space” refers to an open position in or on the rigid central component that is designed to accept a sensor. When the oral apparatus is in use, the sensor capture space is occupied by a sensor. The sensor capture space can be formed from a structural element that is separate from the rigid central component, or it can simply be an empty space within the rigid central component designed to accept the sensor.

Thus the invention comprises an apparatus, methods and systems for improved quantification of accelerations and velocities as experienced by a wearer's head. The measured accelerations and velocities may be specifically associated with the maxilla as representative of the larger skull. A strong coupling between the apparatus and the maxilla or upper jaw and/or upper teeth can provide better accuracy in detection of forces acting on the skull. The layer of saliva between the sensor capture component and the user's upper jaw, maxilla, or teeth facilitates stronger coupling via mechanical locking and surface tension.

In some embodiments, the invention includes a sensor or sensors to track the linear and rotational accelerations during a head impact (non-invasively). In some embodiments, a rigid central capture component facilitates coupling or attachment of the sensor to a bony aspect of the mouth such as the teeth or maxilla (or both). In some embodiments, the sensor is incorporated into an oral appliance or apparatus that can feature a rigid central component. In some embodiments, the oral apparatus is custom made for the wearer's mouth.

The oral apparatus can include various electronic components. Some embodiments include a sensor. Additional components related to the function of the sensor may also be included in the oral apparatus, or they may be remotely positioned to receive signals from the sensor. For example, a power source can be incorporated into a rigid central capture component, or into some other aspect of the oral apparatus. A wire may also be incorporated, for example to connect the sensor with the power source. Electronic components may be placed on a flexible substrate but embedded in the apparatus (or mounted on the apparatus). Or wireless technologies or other hardware or software may be employed. In some embodiments all electronic components may be placed together in or on the oral apparatus, while in other embodiments separate electronic components can occupy different capture spaces in (or on) the oral apparatus. For example, a sensor may be placed on one side of a rigid central component, while a power source is placed on the opposite side of the rigid central component, with a wire connecting the sensor to the power source. In some embodiments the design placement of elements in the capture component is intended to streamline the apparatus. That is, it may be desirable to reduce or minimize the profile, for example in a low-profile custom apparatus.

In one aspect, the invention comprises an apparatus or appliance with a rigid mechanical coupling between the teeth and a streamlined sensor. Coupling a sensor to the upper jaw (maxilla) can more accurately and precisely measure skull kinematics. A strong, rigid coupling mechanism, similar to a dental-appliance based approach (optionally using modified materials) can provide a better coupling. The coupling is facilitated by a rigid central component of the oral apparatus. The rigid central component may serve as a sensor capture component. An oral apparatus may further comprise a full or partial mouthguard.

Thus in some embodiments, the invention comprises an oral apparatus for rigidly coupling a sensor or plurality of sensors to the maxilla (bony upper jaw). The apparatus may be custom made for the mouth of a specific person (similar to a molded dental retainer).

In another aspect, the invention apparatus comprises a low profile to minimize discomfort and/or disruption of speech for the wearer. A low profile can also facilitate easier breathing for the user (as compared to a high profile mouthguard). Thus some embodiments comprise a smaller sensor than has previously been used in devices designed to measure impacts. A sensor may be smaller in any one dimension or multiple dimensions and may also be shaped optimally to minimize the profile of the apparatus in the mouth. As improvements in sensor technology allow for smaller sensors and better incorporation of wireless transmitters or other technologies, an oral apparatus can also have a lower profile and minimize interference with speech and other functions. Generally there are surface areas inside the mouth/palate for which contact with an oral apparatus can be minimized, so as to interfere less with speech, breathing, etc.

As sensor technology improves, sensors can be designed to occupy smaller spaces. Thus in some embodiments, the apparatus comprises a sensor that is shorter, narrower, and a good deal thinner than previously used sensors, so as to reduce the bulk of the oral appliance overall and allow more freedom and comfort.

The apparatus may further comprise a rigid central capture component, which serves as a custom unit for capturing the sensor. The central capture component can be a rigid material. The rigid central component of the oral apparatus may comprise at least a sensor. The rigid central component may partially or fully enclose the sensor in a capture space or provide an attachment point or mounting hardware for the sensor. The rigid central component may be formed in a variety of shapes, including custom-fit to an individual user's mouth, for example in a shape similar to a dental retainer configuration. The rigid central component may comprise acrylic or other rigid or non-resilient materials. For example, a rigid central component can comprise self-polymerizing acrylic. Thus, in some embodiments, the rigid central capture component may have custom dimensions to optimally fit a user's mouth.

In some embodiments of an oral apparatus, the sensor is incorporated by sliding or wedging it into a designated slot or space designed to accommodate the sensor. In some embodiments, a sensor capture space can be positioned laterally, to be coupled to the hard palate next to the upper rear teeth. In other embodiments, a sensor can be positioned centrally, anteriorly, or otherwise, to accommodate needs or preferences of the user.

The illustrative examples provided with this disclosure are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative examples but, like the illustrative examples, should not be used to limit the present disclosure.

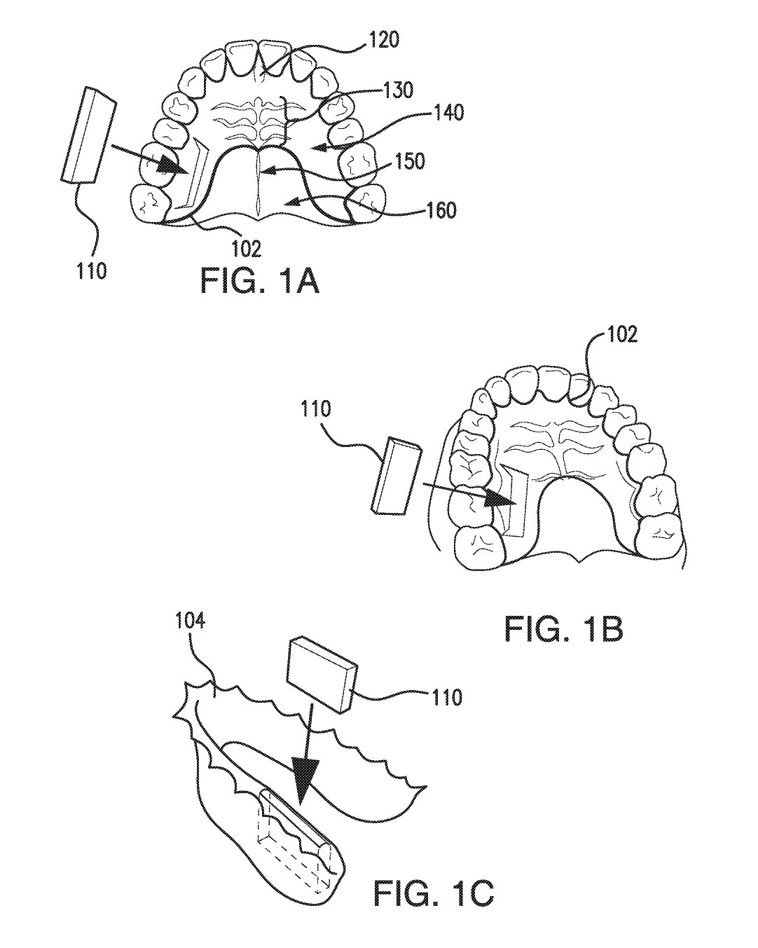

For example, FIG. 1 shows the inferior aspect of the upper jaw in FIGS. 1A and 1B, with bold outlining 102 to show examples of anatomical areas where a rigid central component 104, comprising a sensor 110, can contact the maxillary anatomy, i.e., palate or upper jaw. For reference, anatomical structures are labeled, including incisive papilla 120, palatine rugae 130, the hard palate 140, median palatine suture 150, and transverse suture 160. FIG. 1C illustrates an embodiment of a rigid central component 104 with a sensor 110 placed laterally. The sensor 110 can be completely enclosed in the rigid central component 104, or it can be mountable or otherwise attachable to the rigid central component 104.

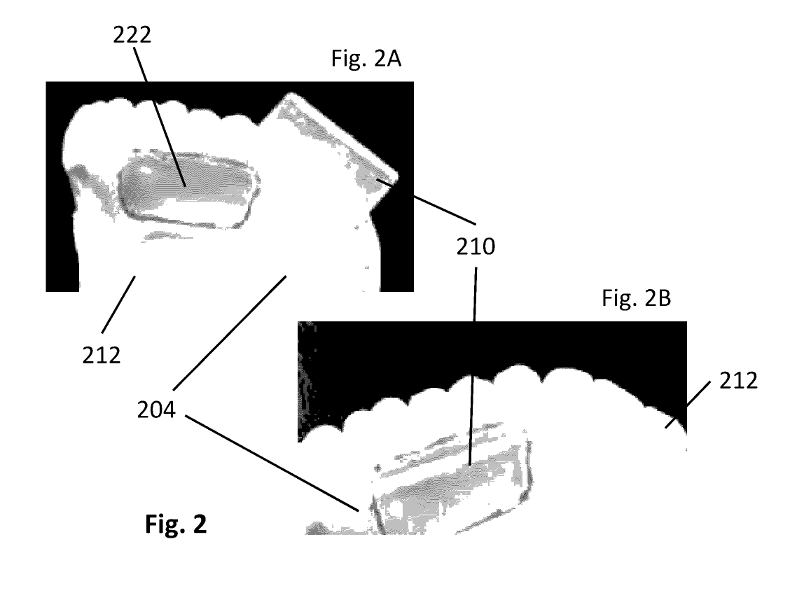

FIG. 2 shows two photographs of a prototype custom rigid central component with sensor capture space according to one embodiment of the invention, with a sensor shown next to the rigid central component in FIG. 2A and the sensor captured in the rigid central component in FIG. 2B. Specifically, FIGS. 2A and 2B show photographs of an embodiment having a sensor 210 placed laterally into a rigid central component 204. Both components are coupled to a mold 212 of a user's or wearer's maxilla and upper teeth. In FIG. 2A, the sensor 210 is near but not placed in the capture space 222 within the rigid central component 204. In FIG. 2B, the sensor is placed in the capture space.

In this embodiment shown in FIGS. 2A and 2B, the sensor slides into the capture space within the rigid central component, and it can be removable. In some embodiments, the sensor may slide into place and latch securely. In some embodiments, the proper placement of the sensor into the capture space can be felt mechanically and/or can be heard with an audible click/snap. For example the mechanism may include a lip at the edge of the capture space to prevent angular displacement. Or other mechanisms may be used to secure the sensor to the capture component.

In other embodiments, an oral apparatus may include a hard or rigid central component, a sensor component incorporated into the rigid central component, and an outer component made of a more resilient material. The resilient outer component can be adhered or bonded to the rigid central component in a temporary or permanent manner.

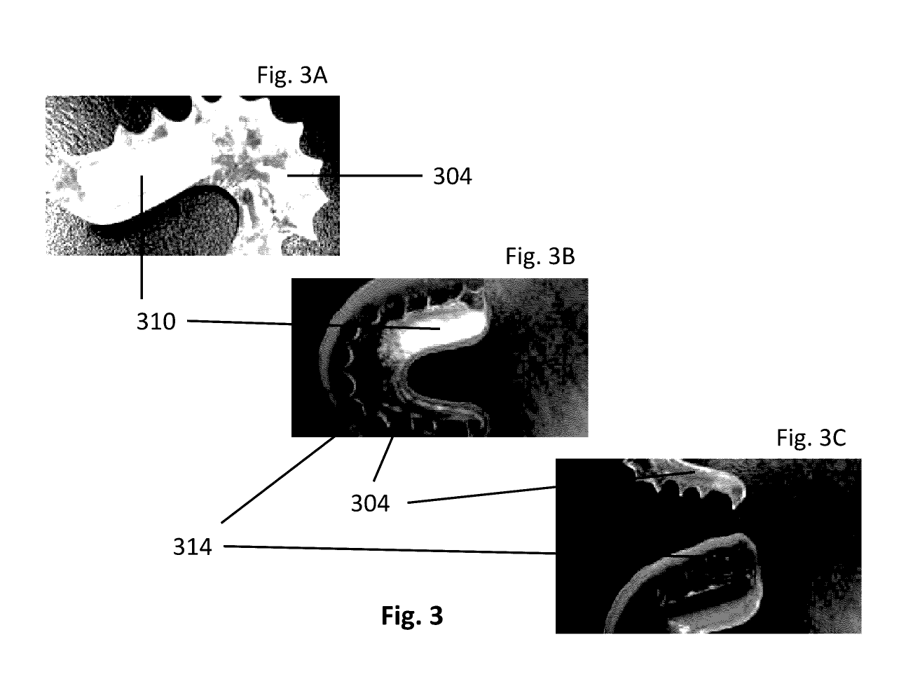

FIG. 3 shows photographs of potential prototype components according to one embodiment of an oral apparatus. FIG. 3A shows the rigid central capture component 304 with sensor 310 in place (in the capture space), FIG. 3B shows the resilient outer (surrounding) component 314 with the rigid central component 304 fitted inside, and FIG. 3C shows parts of the separate resilient 314 and rigid 304 components.

A resilient outer component can be durable but resilient and may bond temporarily or permanently with a rigid central component. In some embodiments, a resilient outer component may be made of materials similar to traditional mouthguards, or it can be more or less resilient than traditional mouthguards. A stiffer mouthguard can discourage chewing which is detrimental to the sensor components. Lack of direct coverage with biting surface of teeth between resilient and nonresilient components can reduce inadvertent teeth impacts which lead to higher apparent accelerations and are thus associated with false impacts.

In some embodiments, a bite guard portion of the outer component may be made of various composite materials. A bite guard component may be an adhered or composite component or a piece that slips over like a separate sports mouthguard to cover the central rigid component of the apparatus. The oral apparatus generally may comprise hard and soft components for coupling but also for impact protection. In some embodiments, the outer component may include a small grab bar for the purpose of removing and inserting the mouthguard. In some embodiments, a grab bar can feature a ball clasp for firmer retention and to facilitate easier removal of the apparatus. A grab bar and ball clasp, or another mechanism to facilitate removal, can be necessary due to the strong coupling created between the apparatus and the upper jaw.

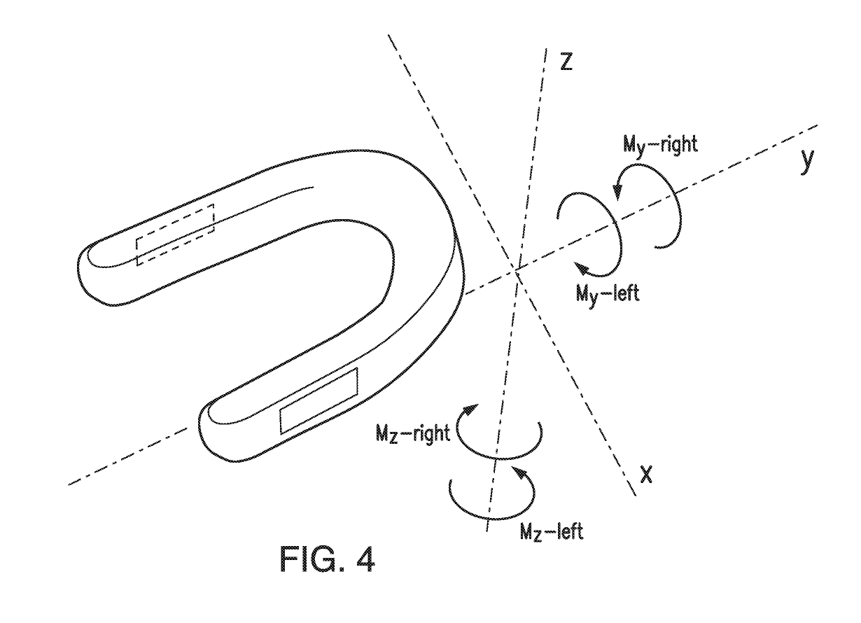

FIG. 4 illustrates the axes of motion with respect to a rigid custom capture component. A rigid custom sensor capture component may be pre-stressed with Bending Moment M_{z} to apply a cantilever force to the inside and/or inferior aspect of the teeth or bone to maintain pressure sufficient to hold the sensor against the teeth or maxillary bone. Alternatively, a sensor capture component may instead be pre-stressed with Torsional Moment M_{y} such that it applies a force to the inside and/or inferior aspect of the teeth or bone to maintain pressure that can hold the sensor against the teeth or maxillary bone. Or both types of pre-stressing may be applied to the capture component. FIG. 4 illustrates alignment of a sensor and capture component with respect to the axes. The methods used to pre-stress depend on the materials of the rigid capture component.

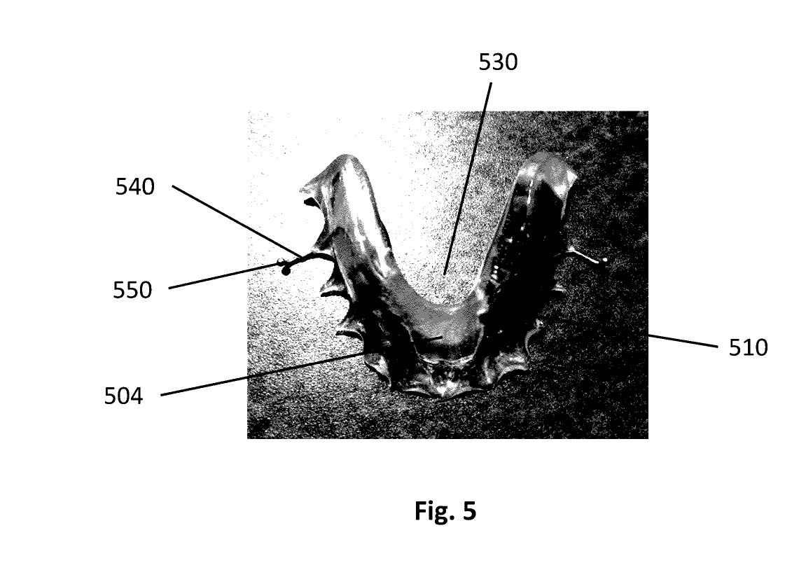

FIG. 5 shows a photograph of a prototype rigid central capture component according to one embodiment, comprising a sensor and other electronic components. The rigid central capture component 504 features an open central aspect 530 that facilitates speech and other functional aspects of the mouth. Embedded in the rigid central component 504 are a sensor 510 and other electronic components. The lateral aspects of the rigid central component 504 feature a grab bar 540 with a ball clasp 550.

In some embodiments, a sensor may be mounted or placed in a medial position on the rigid central capture component. In other embodiments, a sensor may be in a lateral position on the rigid central component. Or a plurality of sensors may occupy various positions from medial to lateral on the rigid central component. The rigid central component may have a single cross-section profile (similar to FIG. 4) or may vary along its length such that it has multiple cross sections, as in FIG. 5. Likewise, the sensor can be any shape or size, including curved, tapered, or other configurations, to fit into the rigid central component.

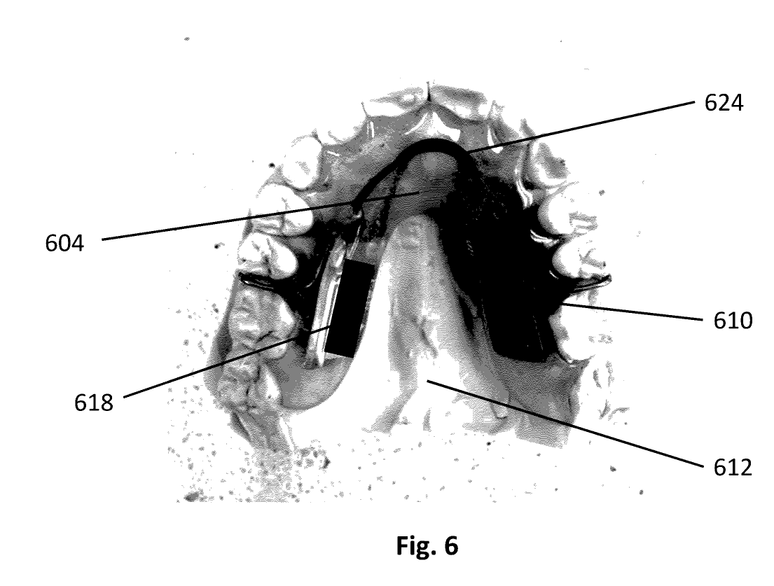

FIG. 6 shows a photograph of the embodiment prototype from FIG. 5, coupled with a mold of the wearer's upper jaw and teeth. In this view, which corresponds to the inferior aspect of the maxilla as represented by the mold 612, the rigid central component 604 can be seen to encompass a sensor 610, a power source 618 (e.g., a battery), and a wire 624 connecting the sensor 610 to the power source 618.

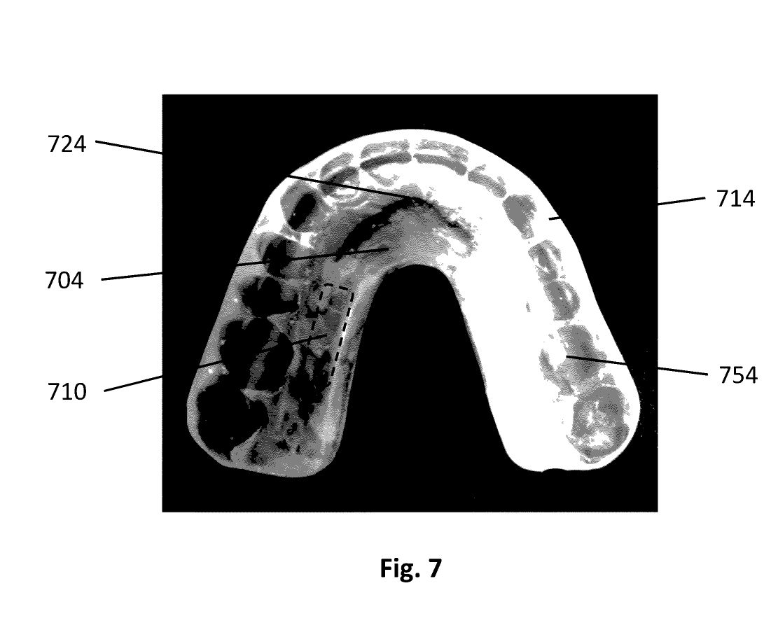

FIG. 7 shows a photograph of prototype resilient outer component embodiment, coupled with a rigid central component. The resilient outer component 714 encompasses a rigid central component 704 comprising electronic components such as a sensor 710 and a wire 724. Other electronic components are present but not visible. In this prototype example, the resilient outer component includes custom indentions for the wearer's teeth 754. Thus some embodiments include both a rigid central component and a resilient outer component.

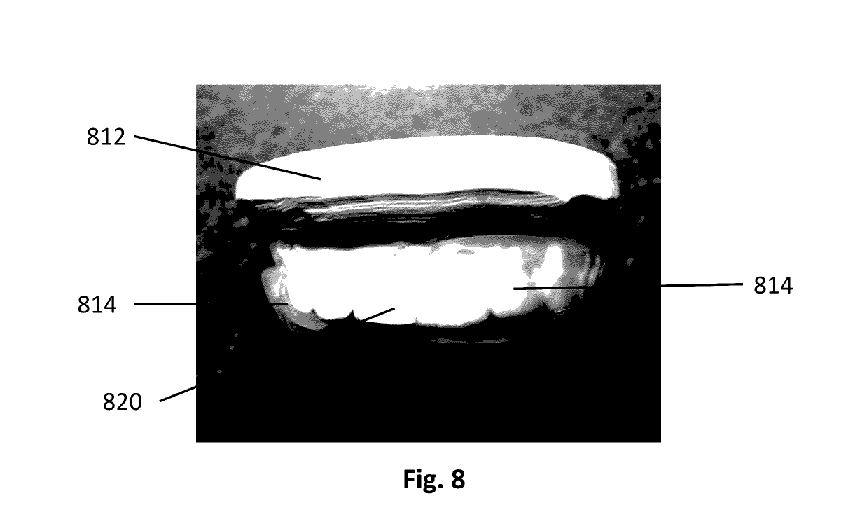

FIG. 8 shows a photograph of an embodiment similar to the prototype in FIG. 7 but placed on a mold of the user's upper jaw and having part of the outermost aspect of the resilient component removed to show contrast and thickness. In this anterior view, the oral apparatus is mounted on a mold 812 of the user's upper jaw. The resilient outer component 814 is the only aspect of the oral apparatus that is visible, with a section cut away to expose the mold of the teeth 820. This view allows observation of the thickness of the resilient component 814, which can be varied according to the physical activities and preferences of the user.

Various configurations are possible, for example to allow for removability or permanent placement of the sensor in the oral apparatus. In some oral apparatus embodiments, the sensor capture space may be altered such that it creates a spring loaded fit (slight interference fit) that grips and holds the sensor within the mouth, such that it does not fall out. Alternatively, the capture space may be altered so that it includes a tensioned soft component (similar to a rubber band) that holds the sensor in the hard part of the apparatus. Or other latching mechanisms are optional. In some embodiments a capture space can include a lip designed to prevent angular displacement and a tab designed to prevent distal migration. In some embodiments, a sensor may click into the capture space.

If the sensor does not need to be removable from the apparatus, it may be entirely encased within the rigid central component of the apparatus. Some oral apparatus embodiments can be disassembled and reassembled and therefore are reusable. Other embodiments can be considered disposable, for example if disassembly is needed but the oral apparatus is destroyed when it is disassembled. In some embodiments, an oral apparatus can be designed so that many of the surfaces adjacent to teeth are soft to avoid high frequency components from contact creating unrealistic accelerations in the sensor (also known as “clacking”).

Methods of using an oral apparatus include the user wearing the oral apparatus during sports or other activities. Methods for use also include data collection, which could be via wired download, wireless technologies, or other means. Power could be provided to the sensor via wired or inductive means, or other technologies.

Various configurations can also provide permanent or temporary coupling of a rigid central component to a resilient outer component. For example, an adhesive can be applied to permanently fix the two components together. Or the placement of a rigid central component into the mouth, followed by the further placement of a resilient outer component, can create a strong but detachable coupling by virtue of the saliva present in the mouth.

Embodiments of the oral apparatus can include a sealing aspect, for example to protect the electronic components from moisture and/or to prevent electronics from being impacted by flexing, biting, or chewing by the user. In some embodiments, the apparatus can be hermetically sealed with an additional hard non-corrosive material. In some embodiments, the electronic components can be housed in a capture space which is coated and sealed with a non-corrosive protective (e.g., silicone, acrylic) spray prior to embedding in (or otherwise attaching to) the rigid central capture component. Other methods for water-proofing with non-corrosive sealants are also possible.

In some embodiments, the apparatus may include a variety of resilient and/or non-resilient materials in a multitude of combinations. For example, a rigid central capture component of the apparatus may comprise metal, acrylic, or other non-resilient materials, while a surrounding portion of the apparatus may comprise ethylene vinyl acetate (EVA), gel compositions, or other resilient materials for a form and feel similar to a regular mouthguard.

Some embodiments can be prepared by injection molding (e.g., using an injection-molded elastomer). Other embodiments can be vacuum-formed or prepared using other technologies. Preferred embodiments avoid curing processes that involve heat (i.e., heat-cured materials), as the electronic components can be affected. In some cases, it can be desirable to limit the contact area with the palate to the surfaces anterior to the first molar. In some embodiments, a custom rigid central component can be compatible with existing commercially available so-called “boil-and-bite” mouthguards. That is, the rigid central component can be placed in the mouth prior to using the formative steps as instructed by the manufacturer for such mouthguards.

In some embodiments, a custom sensor capture component can provide optimal rigidity in an apparatus that applies consistent force to the sensor to maintain its position against the teeth. Pre-stressing the rigid capture component may provide a baseline force/dynamic reading when the mouth piece is in properly. In some embodiments, pre-stressing may also facilitate the ability of the apparatus to provide an alert if during the activity, the player stops wearing it properly, or if the sensor becomes damaged.

The sensor(s) may be capable of quantifying peak linear and rotational accelerations in three translational axes and about three rotational axes, similar to the HITS system in football, as well as complete time history of the linear and rotational accelerations measured about the three translational axes and three rotational axes. In some embodiments, for example where certain sports do not require a mouthpiece and the wearer desires reduced or no external visibility, an apparatus can be completely contained in the mouth such that it is not visible externally. Likewise, where a resilient outer component is included, the thickness and color of such an outer component can be varied to suit the activity and the wearer or user.

Reduced sensor size, streamlined shape, better placement, and configuration within a larger custom apparatus made of a combination of resilient and non-resilient materials can provide a more accurate measurement of the accelerations and velocities of the wearer's head.

The foregoing description of certain examples, including illustrated examples, has been presented only for the purpose of illustration and description and is not intended to be exhaustive or to limit the disclosure to the precise forms disclosed. Numerous modifications, adaptations, and uses thereof will be apparent to those skilled in the art without departing from the scope of the disclosure.

### EXAMPLES

### Example 1

A small form factor sensor system is incorporated into a rigid central component, without an additional outer component, for head force and impact studies in sports not requiring mouth gear. The small form factor is shorter, narrower, and thinner than previous designs intended for use on the skin. Appliances are tested with variable placement of a mouth-based sensor into a mouthguard or other oral appliance so that it couples to the upper jaw, taking advantage of additional bonding strength provided by the layer of saliva between the appliance and the maxilla, for measurement of accelerations and velocities. A prototype is shown in FIG. 5-6.

### Example 2

A small form factor sensor system is housed in a rigid central component, which is further overlayed with a resilient outer component, to simulate implementation into a mouthguard-based system for initial studies. This example can combine existing mouthguard systems with the rigid central component comprising a sensor. A prototype of this combination is shown in FIGS. 7-8.

1. An oral apparatus for coupling a sensor to bony portions of the upper jaw and/or teeth, comprising a rigid central component and a sensor.

2. The apparatus of claim 1, further comprising a resilient outer component.

3. The apparatus of claim 1, wherein the rigid central component comprises a sensor capture space.

4. The apparatus of claim 1, wherein the rigid central component is conformed with bending moment m_{z} to apply cantilever force to inside and/or inferior aspect of palatine process to keep sensor next to the teeth and/or hard palate.

5. The apparatus of claim 1, wherein the rigid central component is conformed with torsional moment m_{y} to apply a force to inside and/or inferior aspect of teeth or bone to keep sensor in contact with teeth and/or bone.

6. The apparatus of claim 1, wherein the sensor is attached to a medial aspect of the rigid central component or a lateral aspect of the rigid central component.

7. The apparatus of claim 1, wherein the rigid central component couples the sensor to the upper jaw and/or upper teeth.

8. The apparatus of claim 1, further comprising a plurality of sensors or additional electronic components.

9. The apparatus of claim 1, wherein the component arrangement allows for contact of tongue with the central lingual surface of upper jaw.

10. The apparatus of claim 1, comprising a connection with the palatine process via custom fit to the upper jaw and teeth.

11. The apparatus of claim 1, comprising attachments at first or second molars from side to side.

12. The apparatus of claim 8, wherein electronic components are protected by a non-corrosive sealant.

13. The apparatus of claim 1, wherein the apparatus comprises metal, acrylic, or other rigid material in at least one component.

14. The apparatus of claim 1, wherein the apparatus is further surrounded by a resilient component bite guard or bite wings.

15. The apparatus of claim 14, wherein the resilient component covers the rear but not front teeth.

16. The apparatus of claim 1, further comprising a resilient overlay adjacent to the rigid central component.

17. The apparatus of claim 16, wherein the resilient overlay is permanently adhered to the rigid central component.

18. The apparatus of claim 16, wherein the resilient overlay is detachable from the rigid central component.

19. The apparatus of claim 16, wherein the resilient overlay is a mouthguard.

20. The apparatus of claim 1, further comprising a power source.

21. The apparatus of claim 1, wherein the sensor is detachable from the apparatus.

22. A method for manufacturing a custom oral apparatus for coupling a sensor to bony portions of the upper jaw, comprising:
a. making an inverse mold for player,
b. setting a rigid capture component configuration in the mold,
c. incorporating a sensor capture setting into the rigid capture component, and
d. connecting a sensor to a rigid capture component.
